(19) United States
(12) Patent Application Publication
(10) Pub. No.: US 2025/0132041 A1
(43) Pub. Date: Apr. 24, 2025

(54) OUTPUT METHOD, OUTPUT APPARATUS, AND COMPUTER-READABLE STORAGE MEDIUM THAT NON-TRANSITORILY STORES PROGRAM

(71) Applicant: Cardio Intelligence Inc., Tokyo (JP)

(72) Inventors: Yuichi TAMURA, Tokyo (JP); Tomohiro TAKATA, Tokyo (JP); Hirohisa TANIGUCHI, Tokyo (JP)

(21) Appl. No.: 19/001,410

(22) Filed: Dec. 25, 2024

Related U.S. Application Data
(63) Continuation of application No. PCT/JP2023/034080, filed on Sep. 20, 2023.

(30) Foreign Application Priority Data
Nov. 16, 2022 (JP) 2022-183474

(51) Int. Cl. A61B 5/361; A61B 5/00; G16H 50/20

(52) U.S. Cl. CPC A61B 5/361; A61B 5/7203; G16H 50/20

(57) ABSTRACT

A computer is caused to execute an acquisition step of acquiring a plurality of pieces of fibrillation wave electrocardiogram data including a fibrillation wave from overall electrocardiogram data obtained by measuring temporal changes of an activity potential during an electrical activity of cardiac muscle; and an output step of outputting clear electrocardiogram data from the acquired plurality of pieces of fibrillation wave electrocardiogram data, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed.

### CROSS-REFERENCE TO RELATED APPLICATIONS

The present application is a continuation application of International Application number PCT/JP2023/34080, filed on Sep. 20, 2023, which claims priority under 35 U.S.C § 119(a) to Japanese Patent Application No. 2022-183474, filed on Nov. 16, 2022, contents of which are incorporated herein by reference in their entirety.

### BACKGROUND OF THE INVENTION

The present invention relates to an output method, an output apparatus, and a computer-readable storage medium that non-transitorily stores a program for analyzing electrocardiograms.

For diagnosis of cardiac diseases such as paroxysmal atrial fibrillation, it is necessary to inspect an electrocardiogram obtained by measurement performed for a long time, in some cases. Since an electrocardiogram includes as many as 100,000 waveforms for a 24-hour period, it is difficult especially for a doctor who is not a medical specialist of the circulatory system to diagnose such cardiac diseases on the basis of electrocardiogram data. Japanese Unexamined Patent Application Publication (Translation of PCT Application) No. 2019-502426 discloses that, in a case where anomalous cardiac beats are sensed in a monitored electrocardiogram (ECG) sample, the electrocardiogram sample is flagged in order for a medical specialist to perform an emergency close examination.

In the technology disclosed in Japanese Unexamined Patent Application Publication (Translation of PCT Application) No. 2019-502426, the electrocardiogram sample flagged in order to perform an emergency close examination is transmitted to a medical specialist of the circulatory system for a close examination. On the other hand, there are cases where an electrocardiogram waveform of a patient having atrial fibrillation includes clear fibrillation waves, which are characteristics of atrial fibrillation, and cases where the electrocardiogram includes unclear fibrillation waves. Conventionally, it had not been possible for a medical specialist who is not a cardiologist to diagnose whether or not a patient is having atrial fibrillation in a case where the medical specialist sees an atrial fibrillation waveform with unclear fibrillation waves.

### BRIEF SUMMARY OF THE INVENTION

The present invention has been made in view of these matters, and an object thereof is to provide an output method, an output apparatus, and a computer-readable storage medium that non-transitorily stores a program for making it possible for a doctor to grasp an electrocardiogram waveform which is useful for the doctor to diagnose whether or not a patient is having atrial fibrillation.

An output method according to a first aspect of the present invention includes: a first acquisition step of acquiring a plurality of pieces of fibrillation wave electrocardiogram data including a fibrillation wave from overall electrocardiogram data obtained by measuring temporal changes of an activity potential during an electrical activity of cardiac muscle; a second acquisition step of acquiring clear electrocardiogram data from the plurality of pieces of fibrillation wave electrocardiogram data acquired at the first acquisition step, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed; and an output step of outputting the clear electrocardiogram data acquired at the second acquisition step.

An output apparatus according to a second aspect of the present invention includes: a first acquiring section that acquires a plurality of pieces of fibrillation wave electrocardiogram data including a fibrillation wave from overall electrocardiogram data obtained by measuring temporal changes of an activity potential during an electrical activity of cardiac muscle; a second acquiring section that acquires clear electrocardiogram data from the plurality of pieces of fibrillation wave electrocardiogram data acquired by the first acquiring section, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed; and an output section that outputs the clear electrocardiogram data acquired by the second acquiring section.

A computer-readable storage medium that non-transitorily stores a program according to a third aspect of the present invention causes a computer to execute: a first acquisition step of acquiring a plurality of pieces of fibrillation wave electrocardiogram data including a fibrillation wave from overall electrocardiogram data obtained by measuring temporal changes of an activity potential during an electrical activity of cardiac muscle; a second acquisition step of acquiring clear electrocardiogram data from the plurality of pieces of fibrillation wave electrocardiogram data acquired at the first acquisition step, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed; and an output step of outputting the clear electrocardiogram data acquired at the second acquisition step.

### BRIEF DESCRIPTION OF THE DRAWINGS

FIG. 1 is a figure for explaining a summary of an electrocardiogram output system S according to an embodiment.

FIG. 2 is a diagram depicting the configuration of an output apparatus.

FIG. 3 is a figure depicting examples of overall electrocardiogram data and divided electrocardiogram data.

FIG. 4A is a figure depicting an example of an electrocardiogram including fibrillation waves.

FIG. 4B is a figure depicting an example of an electrocardiogram including fibrillation waves.

FIG. 5A depicts an example of electrocardiogram data in which fibrillation waves are clear.

FIG. 5B depicts an example of electrocardiogram data in which fibrillation waves are clear.

FIG. 5C depicts an example of electrocardiogram data in which fibrillation waves are clear.

FIG. 6A depicts an example of electrocardiogram data in which fibrillation waves are included but are unclear.

FIG. 6B depicts an example of electrocardiogram data in which fibrillation waves are included but are unclear.

FIG. 6C depicts an example of electrocardiogram data in which fibrillation waves are included but are unclear.

FIG. 7A depicts an example of electrocardiogram data in which fibrillation waves are included but are unclear.

FIG. 7B depicts an example of electrocardiogram data in which fibrillation waves are included but are unclear.

FIG. 8 is a figure depicting an example of output of clear electrocardiogram data by an output section.

FIG. 9 depicts another example of output of clear electrocardiogram data to a doctor's terminal by the output section.

FIG. 10 depicts another example of output of clear electrocardiogram data to the doctor's terminal by the output section.

FIG. 11 is a flowchart depicting a processing procedure of generation of a display machine learning model by the output apparatus.

FIG. 12 is a flowchart depicting a processing procedure of output of clear electrocardiogram data by the output apparatus.

### DETAILED DESCRIPTION OF THE INVENTION

Hereinafter, the present disclosure will be described through exemplary embodiments, but the following exemplary embodiments do not limit the invention according to the claims, and not all of the combinations of features described in the exemplary embodiments are necessarily essential to the solution means of the invention.

### Summary of Electrocardiogram Output System S

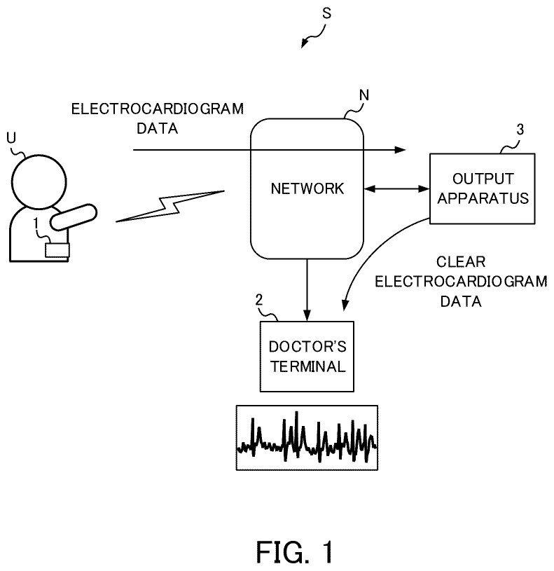

FIG. 1 is a figure for explaining a summary of an electrocardiogram output system S according to the present embodiment. The electrocardiogram output system S is a system for making it easier for a doctor to use an electrocardiogram of a patient U who may be having atrial fibrillation, and diagnose the patient. The electrocardiogram output system S includes an electrocardiograph 1, a doctor's terminal 2, and an output apparatus 3.

For example, the electrocardiograph 1 is a Holter electrocardiograph attached to the patient U. The electrocardiograph 1 generates overall electrocardiogram data obtained by measuring temporal changes of an activity potential during an electrical activity of cardiac muscle of the patient U. The electrocardiograph 1 transmits the generated overall electrocardiogram data to the output apparatus 3 via a network N including a wireless communication line. Time information representing the measurement time of the overall electrocardiogram data is associated with the overall electrocardiogram data. For example, the overall electrocardiogram data generated by the electrocardiograph 1 may be passed on to the output apparatus 3 using a storage medium, without using the network N.

The doctor's terminal 2 is a terminal used by a doctor, and, for example, includes a display apparatus and a computer. The doctor's terminal 2 outputs, on the display apparatus, a waveform image based on electrocardiogram data which is part of the overall electrocardiogram data generated in the electrocardiograph 1, and received from the output apparatus 3.

For example, the output apparatus 3 is a server. The output apparatus 3 outputs information for assisting diagnosis by a doctor as to whether or not the patient U is having atrial fibrillation in her/his heart. The output apparatus 3 receives the overall electrocardiogram data of the patient U from the electrocardiograph 1 or the doctor's terminal 2. The output apparatus 3 generates a plurality of pieces of divided electrocardiogram data obtained by dividing the received overall electrocardiogram data. The output apparatus 3 acquires a plurality of pieces of fibrillation wave electrocardiogram data in which fibrillation waves unique to atrial fibrillation are included, from the plurality of pieces of divided electrocardiogram data.

The output apparatus 3 sub-divided fibrillation generates wave electrocardiogram data obtained by dividing fibrillation wave electrocardiogram data into electrocardiograms each corresponding to a predetermined time unit. The length of each predetermined time unit is time corresponding to one beat, for example. The output apparatus 3 reads out, from a storage section, a trained display machine learning model (equivalent to a first machine learning model) for classifying a plurality of pieces of electrocardiogram data including fibrillation waves into (i) electrocardiogram data in which fibrillation waves are clear and (ii) electrocardiogram data in which fibrillation waves are included but are unclear. The machine learning model can have any internal configuration, and, for example, is configured using a CNN (Convolutional Neural Network, convolutional neural network).

The output apparatus 3 inputs the acquired plurality of pieces of sub-divided fibrillation wave electrocardiogram data to the trained display machine learning model, and acquires clear electrocardiogram data output by the display machine learning model as electrocardiogram data in which fibrillation waves are clear. In examples in the present specification, the display machine learning model outputs, as the clear electrocardiogram data, fibrillation wave electrocardiogram data in the acquired plurality of pieces of fibrillation wave electrocardiogram data, the output fibrillation wave electrocardiogram data being electrocardiogram data in which the amplitude of the fibrillation waves is within a predetermined range, a symptom of tachycardia is not observed, and predetermined noise is not superimposed. The output apparatus 3 outputs the acquired clear electrocardiogram data to the doctor's terminal 2. In this manner, the output apparatus 3 can make it possible for the doctor to grasp an electrocardiogram waveform representing grounds for the doctor to decide that the patient U is having atrial fibrillation in her/his heart.

In addition, this example in which the output apparatus 3 is a computer separate from the doctor's terminal 2 is not the sole example. For example, the output apparatus 3 may be included in the computer which is part of the doctor's terminal 2. Hereinbelow, the configuration of and operations performed by the output apparatus 3 are explained in detail.

### Configuration of Output Apparatus 3

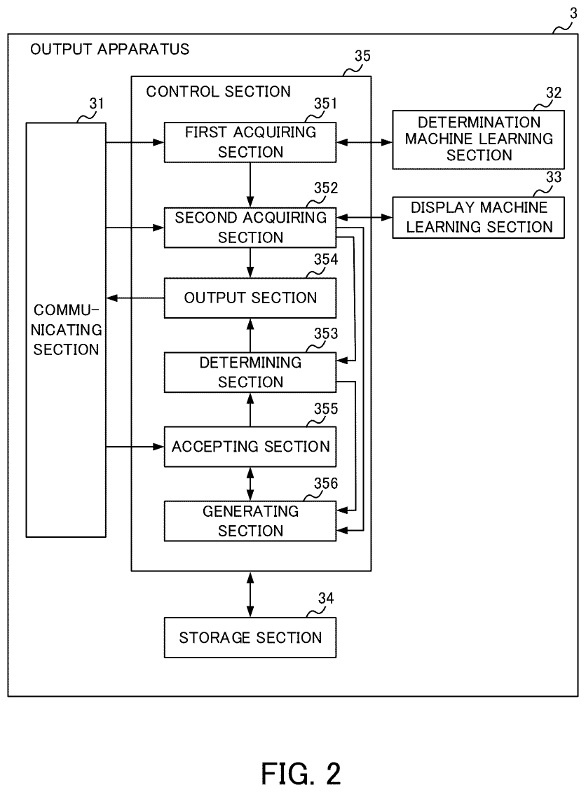

FIG. 2 is a diagram depicting the configuration of the output apparatus 3. The output apparatus 3 has a communicating section 31, a determination machine learning section 32, a display machine learning section 33, a storage section 34, and a control section 35. The control section 35 has a first acquiring section 351, a second acquiring section 352, a determining section 353, an output section 354, an accepting section 355, and a generating section 356.

The communicating section 31 has a communication controller for transmitting and receiving data between the electrocardiograph 1 and the doctor's terminal 2 via the network N. The communicating section 31 notifies the control section 35 of the data received via the network N.

The determination machine learning section 32 functions as a determination machine learning model (equivalent to a second machine learning model) generated by being trained on the basis of electrocardiogram data for training used as teaching data, and can classify an input plurality of pieces of electrocardiogram data into electrocardiogram data in which fibrillation waves are included, and electrocardiogram data in which fibrillation waves are not included. For example, the determination machine learning section 32 includes a processor that executes various types of computation using a CNN, and a memory that stores coefficients of the CNN. The determination machine learning section 32 classifies input electrocardiogram data into electrocardiogram data in which fibrillation waves are included, and electrocardiogram data in which fibrillation waves are not included, and outputs each of them.

The display machine learning section 33 functions as the display machine learning model mentioned above trained such that, in a case where electrocardiogram data in which fibrillation waves are clear, and electrocardiogram data in which fibrillation waves are included but are unclear are input to the display machine learning section 33, the display machine learning section 33 outputs the electrocardiogram data in which fibrillation waves are clear, and does not output the electrocardiogram data in which fibrillation waves are included but are unclear. For example, the display machine learning section 33 includes a processor that executes various types of computation using a CNN, and a memory that stores coefficients of the CNN.

The display machine learning section 33 classifies an input plurality of pieces of electrocardiogram data including fibrillation waves into (i) electrocardiogram data in which fibrillation waves are clear, and (ii) electrocardiogram data in which fibrillation waves are included but are unclear, and outputs each of them for each predetermined time unit. The length of each predetermined time unit is time corresponding to one beat, for example. In addition to classification results of the electrocardiogram data, the display machine learning section 33 may output degrees of clarity representing, with numerical values or the like, the degrees of clarity of fibrillation waves included in the input electrocardiogram data. Details of the degrees of clarity are mentioned later.

This example in which the display machine learning section 33 classifies a plurality of pieces of electrocardiogram data including fibrillation waves into (i) electrocardiogram data in which fibrillation waves are clear, and (ii) electrocardiogram data in which fibrillation waves are included but are unclear, and outputs them is not the sole example. The display machine learning section 33 may function as a display machine learning model that classifies a plurality of pieces of electrocardiogram data including fibrillation waves into electrocardiogram data including heartbeats with clear fibrillation waves at a relatively high proportion, and electrocardiogram data including heartbeats with clear fibrillation waves at a relatively low proportion, and outputs them. For example, such a machine learning model is generated by machine learning using, as teaching data, electrocardiogram data determined by a doctor as including heartbeats with clear fibrillation waves at a relatively high proportion, and electrocardiogram data determined by the doctor as including heartbeats with clear fibrillation waves at a relatively low proportion.

The display machine learning section 33 may output classification results which are obtained by classifying a plurality of pieces of electrocardiogram data including fibrillation waves into electrocardiogram data including heartbeats with clear fibrillation waves at a relatively high proportion, and electrocardiogram data including heartbeats with clear fibrillation waves at a relatively low proportion, and output, along with the classification results, a clear heartbeat index representing, with a numerical value or the like, the proportion at which heartbeats with clear fibrillation waves are included in each of the pieces of electrocardiogram data.

The display machine learning section 33 may use the Fourier transform or the like to classify a plurality of pieces of electrocardiogram data including fibrillation waves into electrocardiogram data including heartbeats with clear fibrillation waves at a relatively high proportion, and electrocardiogram data including heartbeats with clear fibrillation waves at a relatively low proportion, and output at least either of the classified pieces of electrocardiogram data. For example, the display machine learning section 33 may classify electrocardiogram data including frequency components corresponding to clear fibrillation waves at a relatively high proportion as electrocardiogram data including heartbeats with clear fibrillation waves at a relatively high proportion, and classify electrocardiogram data including frequency components corresponding to clear fibrillation waves at a relatively low proportion as electrocardiogram data including heartbeats with clear fibrillation waves at a relatively low proportion.

The storage section 34 includes storage media such as a ROM (Read Only Memory), a RAM (Random Access Memory), and a hard disk. The storage section 34 stores programs to be executed by the control section 35. In addition, the storage section 34 stores various types of data that are required when the control section 35 executes various types of computation.

For example, the control section 35 is a CPU (Central Processing Unit). By executing programs stored on the storage section 34, the control section 35 functions as the first acquiring section 351, the second acquiring section 352, the determining section 353, the output section 354, the accepting section 355, and the generating section 356.

The first acquiring section 351 communicates with the electrocardiograph 1 and the doctor's terminal 2 via the communicating section 31. The first acquiring section 351 acquires, from the doctor's terminal 2, operation information about operation of the doctor's terminal 2 by the doctor. The first acquiring section 351 acquires, from the electrocardiograph 1, overall electrocardiogram data obtained by measuring temporal changes of an activity potential during an electrical activity of cardiac muscle. The first acquiring section 351 acquires a plurality of pieces of fibrillation wave electrocardiogram data in which fibrillation waves are included, from electrocardiogram data included in the acquired overall electrocardiogram data. In addition, the first acquiring section 351 divides the acquired overall electrocardiogram data into a plurality of pieces of divided electrocardiogram data. For example, the first acquiring section 351 generates a plurality of pieces of divided electrocardiogram data obtained by dividing the acquired overall electrocardiogram data into electrocardiograms each corresponding to a 30-second period.

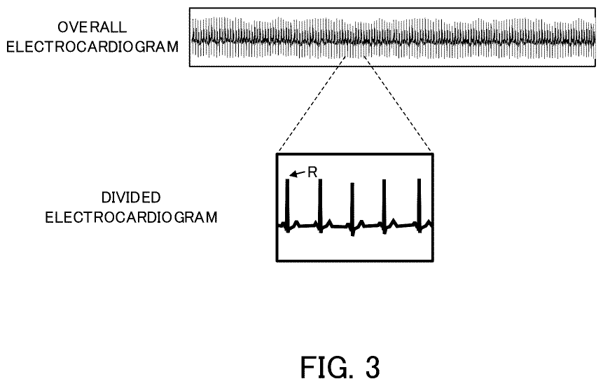

FIG. 3 is a figure depicting examples of overall electrocardiogram data and divided electrocardiogram data. The upper part depicts an overall electrocardiogram included in the overall electrocardiogram data, and the lower part depicts a divided electrocardiogram included in the divided electrocardiogram data. The overall electrocardiogram represents measure results obtained by measuring temporal changes of an activity potential of cardiac muscle of the patient U for predetermined measurement time. For example, the measurement time is 24 hours.

The divided electrocardiogram data is obtained by dividing the overall electrocardiogram data. For example, the divided electrocardiogram data is obtained by dividing the overall electrocardiogram data into electrocardiograms each corresponding to a 30-second period. For example, the divided electrocardiogram data is obtained by dividing the overall electrocardiogram data into electrocardiograms each corresponding to a date and a time period. R in the divided electrocardiogram in FIG. 3 denotes R waves.

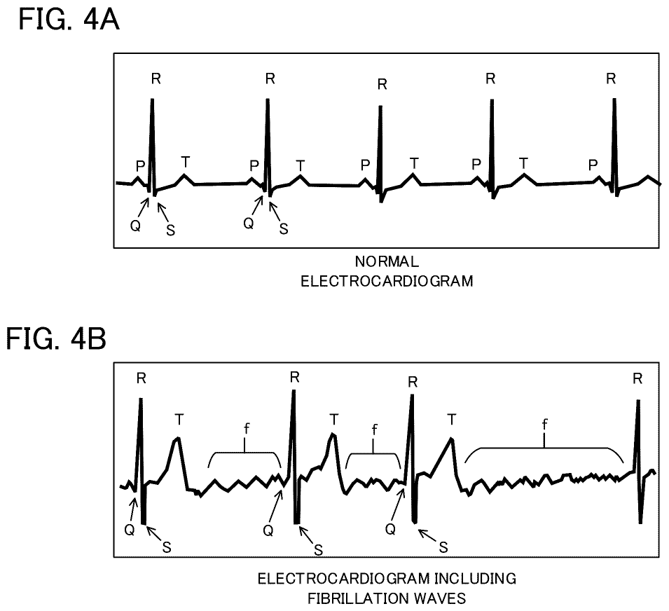

The first acquiring section 351 acquires one or more pieces of fibrillation wave electrocardiogram data in which fibrillation waves are included, from a plurality of pieces of divided electrocardiogram data. FIG. 4A and FIG. 4B are figures depicting examples of electrocardiograms including fibrillation waves. FIG. 4A depicts a normal electrocardiogram. FIG. 4B depicts an example of an electrocardiogram including fibrillation waves. The vertical axis in FIG. 4A represents a potential, and the horizontal axis in FIG. 4A represents time. P, Q, R, S, and T in FIG. 4A denote P waves, Q waves, R waves, S waves, and T waves. As depicted in FIG. 4A, in a normal electrocardiogram, P waves, Q waves, R waves, S waves, and T waves are repeated at predetermined cycles. Fibrillation waves are not included in normal electrocardiogram depicted in FIG. 4A.

f in FIG. 4B denotes fibrillation waves. In the example in FIG. 4B, fibrillation waves are included in the electrocardiogram, and accordingly it can be known that the patient U is having atrial fibrillation in her/her heart. As depicted in FIG. 4B, in an electrocardiogram of a patient having atrial fibrillation, P waves have disappeared, and the cycle of R waves or the like is irregular.

In examples in the present specification, the first acquiring section 351 inputs a plurality of pieces of divided electrocardiogram data to the determination machine learning section 32 that functions as the determination machine learning model (equivalent to the second machine learning model), and acquires, from the determination machine learning section 32, one or more pieces of fibrillation wave electrocardiogram data output as divided electrocardiogram data in which fibrillation waves are included. The first acquiring section 351 outputs the acquired one or more pieces of fibrillation wave electrocardiogram data to the second acquiring section 352.

### Acquisition of Electrocardiogram Data in Which Fibrillation Waves are Clear

The second acquiring section 352 acquires clear electrocardiogram data in which fibrillation waves are clear from one or more pieces of fibrillation wave electrocardiogram data acquired by the first acquiring section 351. More specifically, the second acquiring section 352 generates sub-divided fibrillation wave electrocardiogram data obtained by dividing fibrillation wave electrocardiogram data into electrocardiograms each corresponding to a predetermined time unit. The length of each predetermined time unit is time corresponding to one beat, for example. The second acquiring section 352 inputs one or more pieces of sub-divided fibrillation wave electrocardiogram data to the display machine learning section 33 that functions as the display machine learning model, and acquires, from the display machine learning section 33, electrocardiogram data (hereinbelow, also referred to as clear electrocardiogram data) output as electrocardiogram data in which fibrillation waves are clear.

In examples in the present specification, electrocardiogram data in which fibrillation waves are clear means electrocardiogram data in which (1) the amplitude of fibrillation waves is within a predetermined range, (2) a symptom of tachycardia is not observed, and (3) fibrillation waves on which predetermined noise is not superimposed are included. The predetermined range of the amplitude in (1) is a range greater than 0.05 mV and smaller than 0.5 mV. Preferably, the predetermined range of the amplitude is a range greater than 0.05 mV and equal to or smaller than 0.25 mV. Tachycardia in (2) is a state where the intervals between R waves is smaller than 400 milliseconds, or a state where the heart rate is higher than 150 bpm.

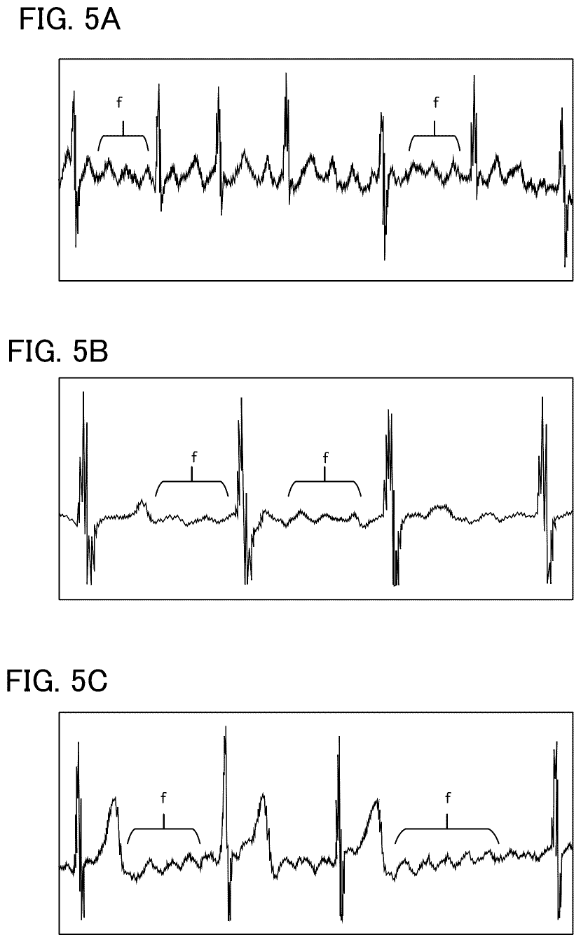

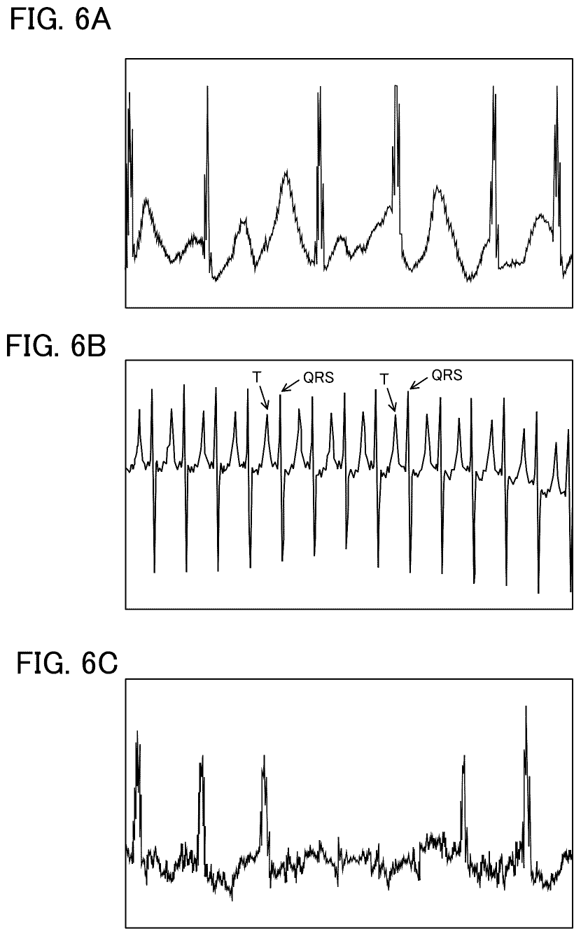

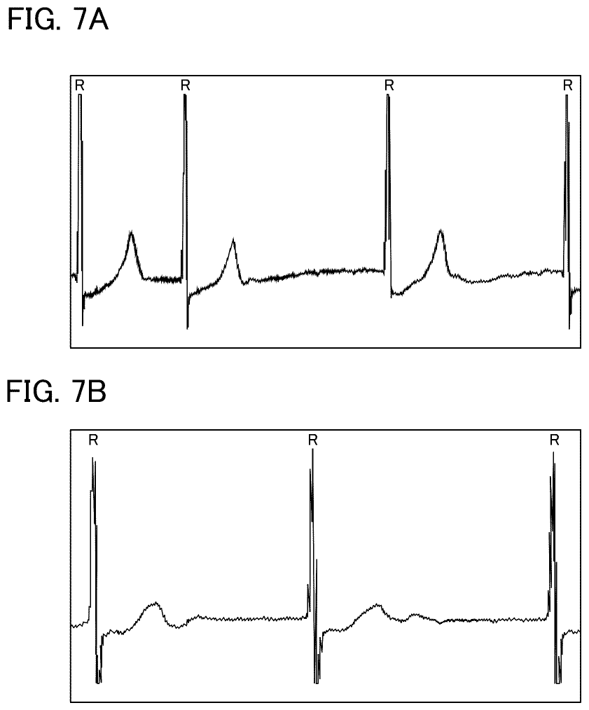

The predetermined noise in (3) is noise with a frequency which is higher than the upper limit value or lower limit value of the frequency range (5 Hz to 10 Hz) of fibrillation waves, and with amplitude which is equal to or greater than predetermined amplitude. For example, the predetermined amplitude is equal to or greater than 0.05 mV, but may be equal to or greater than 0.25 mV. FIG. 5 to FIG. 7 depict examples of criteria for deciding whether or not fibrillation waves are clear. FIG. 5A to FIG. 5C depict examples of electrocardiogram data in which fibrillation waves are clear. FIG. 6A to FIG. 6C, and FIG. 7A and FIG. 7B depict examples of electrocardiogram data in which fibrillation waves are included but are unclear. All the fibrillation waves denoted by f in FIG. 5A to FIG. 5C are depicted clearly in the electrocardiogram data in the figures.

FIG. 6A depicts an example in which fibrillation waves are unclear. In the electrocardiogram data in FIG. 6A, fibrillation waves with amplitude in the predetermined range mentioned above cannot be recognized, and are unclear. In the electrocardiogram data depicted in FIG. 6B, a symptom of tachycardia with a heart rate exceeding 150 bpm is observed. As depicted in FIG. 6B, since T waves (T in FIG. 6B) and QRS waves (QRS in FIG. 6B) are almost continuous in electrocardiogram data in which a symptom of tachycardia is observed, there are few areas where fibrillation waves between T waves and QRS waves can easily be recognized. Because of this, in electrocardiogram data in which a symptom of tachycardia is observed, fibrillation waves are unclear. In the electrocardiogram data depicted in FIG. 6C, the predetermined noise mentioned above is superimposed on fibrillation waves, and accordingly the fibrillation waves are unclear.

FIG. 7A and FIG. 7B depict other examples of electrocardiogram data in which fibrillation waves are included but are unclear. FIG. 7A and FIG. 7B depict examples of electrocardiogram data in a case where fibrillation waves are weak. As depicted in FIG. 7A and FIG. 7B, in a case where the amplitude of fibrillation waves is weak, fibrillation waves having amplitude in the predetermined range mentioned above cannot be recognized, and fibrillation waves are unclear. At this time, the intervals between R waves are irregular.

When acquiring clear electrocardiogram data output by the display machine learning section 33, the second acquiring section 352 may acquire the degrees of clarity of clear electrocardiogram data along with the clear electrocardiogram data. For example, the degrees of clarity have values that increase as the amplitude of fibrillation waves increases. The degrees of clarity have values that increase as the intervals between R waves increase. The degrees of clarity have values that increase as the levels of noise lower. The second acquiring section 352 may acquire, as the degrees of clarity, scores that are output by the determination machine learning section 32, and are for determining whether or not fibrillation waves are included.

In addition, the second acquiring section 352 may acquire an index related to the variance of RR intervals (heartbeats) as the degrees of clarity. For example, the second acquiring section 352 may input a plurality of pieces of divided electrocardiogram data to the determination machine learning section 32, and, as the degrees of clarity and along with fibrillation wave electrocardiogram data in which fibrillation waves are included, acquire an index output by the determination machine learning section 32 and related to the variance of RR intervals of the fibrillation wave electrocardiogram data. In addition, the second acquiring section 352 may acquire the degrees of clarity as combinations of a plurality of indices obtained by combining a score for determining whether or not fibrillation waves are included and an index related to the variance of RR intervals, and so on. Instead of inputting sub-divided fibrillation wave electrocardiogram data to the display machine learning section 33, the second acquiring section 352 may input, to the display machine learning section 33, corrected fibrillation wave electrocardiogram data obtained by removing waves which are not fibrillation waves from the sub-divided fibrillation wave electrocardiogram data. For example, the second acquiring section 352 generates the corrected fibrillation wave electrocardiogram data by removing one or more of P waves, QRS waves, and T waves (see FIG. 4A and FIG. 4B). The second acquiring section 352 inputs the generated one or more pieces of corrected fibrillation wave electrocardiogram data to the display machine learning section 33.

For example, the second acquiring section 352 generates corrected fibrillation wave electrocardiogram data by removing waves having potentials which are equal to or greater than a reference value from an electrocardiogram waveform in which fibrillation waves are included. For example, the reference value is greater than a value that is expected as the peak potential of fibrillation waves. In addition, the second acquiring section 352 identifies a temporal area where a Q wave, an R wave, an S wave, a T wave, or the like is generated. The second acquiring section 352 may generate corrected fibrillation wave electrocardiogram data obtained by removing waves other than fibrillation waves, by setting the potential of an electrocardiogram in the temporal area to zero.

For example, the second acquiring section 352 identifies a temporal area where the potential is equal to or greater than a threshold as a temporal area where an R wave is generated. A temporal area where a Q wave, an S wave, and a T wave are generated can be estimated from temporal areas where an immediately preceding and immediately following R waves are generated. The second acquiring section 352 identifies temporal areas where Q waves, S waves, and T waves are generated, on the basis of an identified plurality of temporal areas where R waves are generated. The second acquiring section 352 may generate corrected fibrillation wave electrocardiogram data by setting the potential of an electrocardiogram in each of the identified temporal areas where Q waves, R waves, S waves, T waves, and the like are generated to zero.

The second acquiring section 352 inputs one or more pieces of corrected fibrillation wave electrocardiogram data obtained by removing respective waves that are not fibrillation waves to the display machine learning section 33, and acquires, from the display machine learning section 33, corrected fibrillation wave electrocardiogram data output as electrocardiogram data in which fibrillation waves are clear. On the basis of the corrected fibrillation wave electrocardiogram data output as electrocardiogram data in which fibrillation waves are clear, the second acquiring section 352 may acquire, as clear electrocardiogram data in which fibrillation waves are clear, fibrillation wave electrocardiogram data before removal of waves which are not fibrillation waves in the corrected fibrillation wave electrocardiogram data.

The second acquiring section 352 may input one or more pieces of fibrillation wave electrocardiogram data to the display machine learning section 33 that functions as the display machine learning model that classifies electrocardiogram data into electrocardiogram data including heartbeats with clear fibrillation waves at a relatively high proportion, and electrocardiogram data including heartbeats with clear fibrillation waves at a relatively low proportion, and outputs the classified electrocardiogram data, and acquire, as clear electrocardiogram data, fibrillation wave electrocardiogram data output as the electrocardiogram data including heartbeats with clear fibrillation waves at a relatively high proportion. Along with the clear electrocardiogram data, the second acquiring section 352 may acquire a clear heartbeat index representing a proportion at which heartbeats with clear fibrillation waves are included in the clear electrocardiogram data.

The determining section 353 determines whether or not there is a signal in a frequency range corresponding to fibrillation waves by performing frequency analysis of the clear electrocardiogram data acquired by the second acquiring section 352. For example, the frequency analysis is Fourier analysis or wavelet analysis. The determining section 353 notifies the output section 354 of a determination result of determination as to whether or not there is a signal in the frequency range corresponding to fibrillation waves.

### Output of Electrocardiogram Data

The output section 354 communicates with the doctor's terminal 2 via the communicating section 31. The output section 354 outputs clear electrocardiogram data acquired by the second acquiring section 352. For example, the output section 354 outputs clear electrocardiogram data to the display apparatus of the doctor's terminal 2. The output section 354 outputs the clear electrocardiogram data acquired by the second acquiring section 352 in a state where image data representing that fibrillation waves are included overlaps temporal areas between a plurality of R waves in the clear electrocardiogram data. The output section 354 outputs, to the display apparatus, the clear wave electrocardiogram data such that the sizes of RR intervals (heartbeats) are equal to or greater than a certain size. This makes it easier for the doctor to visually decide that the RR intervals are irregular.

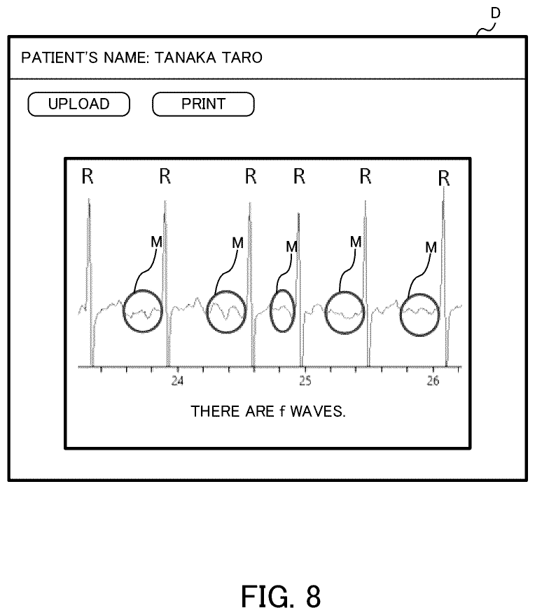

FIG. 8 is a figure depicting an example of output of clear electrocardiogram data by the output section 354. An image depicted in FIG. 8 is output to a display apparatus D of the doctor's terminal 2. R in an electrocardiogram in FIG. 8 denotes R waves. The output section 354 displays image data M representing that fibrillation waves are included in temporal areas between a plurality of R waves. In the example in FIG. 8, oval thick frames are depicted as the image data M.

In a state where there is atrial fibrillation, the intervals between R waves increase or decrease irregularly. Because of this, in the example in FIG. 8, the sizes, in the time direction, of the image data M displayed by the output section 354 differ depending the intervals between R waves. For example, in a case where the interval between R waves is shorter than a predetermined value, the output section 354 displays image data M with a size in the time direction which is a first size. In a case where the interval between R waves is equal to or longer than the predetermined value, the output section 354 displays image data M with a size in the time direction which is a second size. The second size is greater than the first size. In the example in FIG. 8, along with the image data M, the output section 354 outputs a message “There are f Waves” representing that the image data M corresponds to the positions of fibrillation waves.

In a state where there is atrial fibrillation, there are fibrillation waves in almost any time period areas of an electrocardiogram. However, fibrillation waves are not clear at the timings at which the fibrillation waves overlap other waves such as QRS or T waves. In particular, R waves have high peak potentials as compared with other waves, and accordingly fibrillation waves are often clear at positions between a plurality of R waves. Because of this, the output section 354 outputs the image in a state where the image data M representing that fibrillation waves are included overlaps temporal areas between a plurality of R waves. For example, the output section 354 outputs the image in a state where the image data M overlaps temporal areas including intermediate positions between a plurality of R waves. In this manner, the output section 354 can make it easier for the doctor to grasp the temporal areas in which clear fibrillation waves are included.

In a state where there is atrial fibrillation, the disappearance of P waves is observed in an electrocardiogram. It becomes easier to observe a fibrillation wave due to the disappearance of a P wave between a T wave and a QRS wave. Because of this, the output section 354 may identify temporal areas where P waves are supposed to be generated if there is not atrial fibrillation, and display the image data M representing that clear fibrillation waves are included in the identified temporal areas. Since P waves occur before Q waves, the output section 354 may output the image in a state where the image data M overlaps temporal areas in predetermined periods before the timings at which the Q waves start. For example, the predetermined time is determined such that the image data M includes areas of P waves if the P waves have not disappeared.

In addition, in normal electrocardiogram data in which there is not atrial fibrillation, P waves occur between the timings at which T waves end and the timings at which Q waves start (see FIG. 4A). Because of this, the output section 354 may identify the positions of T waves and Q waves in electrocardiogram data, and output the image in a state where the image data M overlaps positions between the timings at which the T waves end and the timings at which the Q waves start.

In addition, the output section 354 may output clear electrocardiogram data determined by the determining section 353 as including signals in the frequency range corresponding to fibrillation waves from the clear electrocardiogram data acquired by the second acquiring section 352. The output section 354 may not output clear electrocardiogram data determined by the determining section 353 as not including signals in the frequency range corresponding to fibrillation waves from the clear electrocardiogram data acquired by the second acquiring section 352. In this manner, in a case where the second acquiring section 352 has acquired, by an error, clear electrocardiogram data in which fibrillation waves are not included, the output section 354 can avoid outputting the clear electrocardiogram data.

The output section 354 outputs, to the display apparatus D of the doctor's terminal 2 or the like, a plurality of pieces of clear electrocardiogram data whose dates or time periods of measurement are different from each other. In this manner, the output section 354 makes it possible for the doctor to evaluate whether there are multiple occurrences of atrial fibrillation events during recording time.

The output section 354 outputs, to the display apparatus D of the doctor's terminal 2 or the like, a plurality of pieces of representative waveform data selected on the basis of the degrees of clarity of fibrillation waves from the plurality of pieces of clear electrocardiogram data. As mentioned above, for example, the degrees of clarity are represented by numerical values representing the degrees of clarity of fibrillation waves of the electrocardiogram data. For example, the output section 354 outputs, as representative waveform data, clear electrocardiogram data in descending order of the degrees of clarity of fibrillation waves acquired by the second acquiring section 352 along with the clear electrocardiogram data and included in the clear electrocardiogram data.

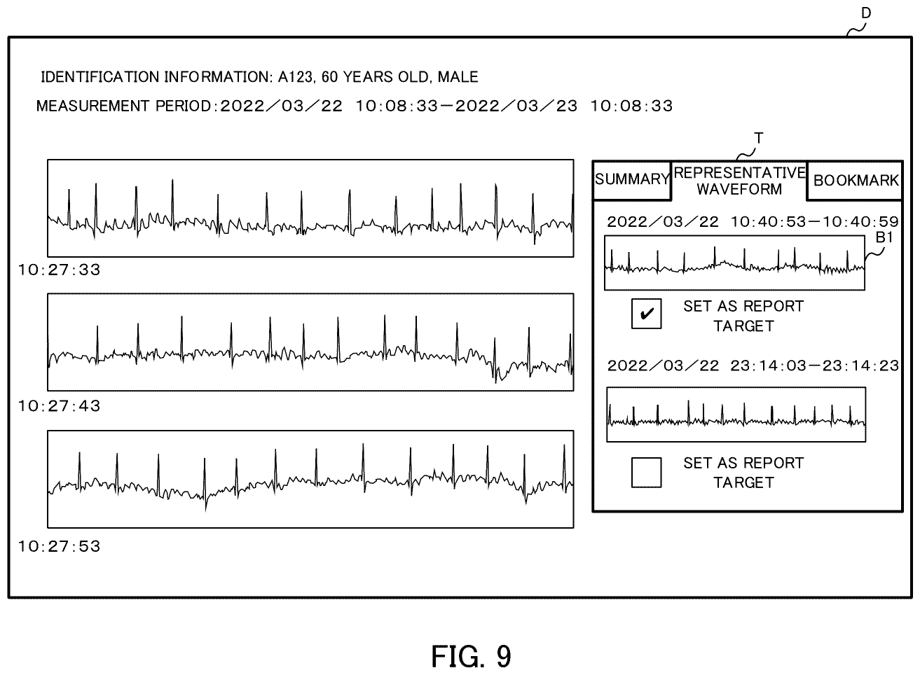

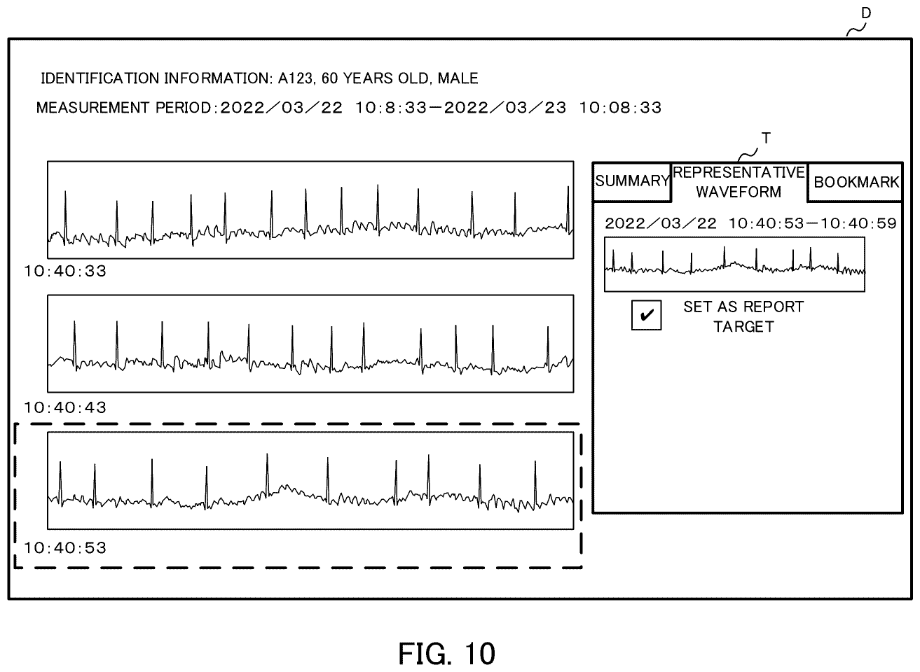

FIG. 9 and FIG. 10 depict other examples of output of clear electrocardiogram data to the doctor's terminal 2 by the output section 354. Images depicted in FIG. 9 and FIG. 10 are output to the display apparatus D of the doctor's terminal 2. The left side of FIG. 9 depicts a plurality of pieces of divided electrocardiogram data corresponding to an ID No. “A123.” Upon acquisition, by the first acquiring section 351 and from the doctor's terminal 2, of operation information representing selection of a representative waveform tab T by the doctor, the output section 354 outputs the plurality of pieces of representative waveform data as depicted on the right side in FIG. 9.

The output section 354 outputs, as selected waveform data, a representative waveform data selected by a user such as the doctor from the plurality of pieces of representative waveform data output to the display apparatus D. In the example in FIG. 9, the output section 354 outputs a checkbox and a character string “Set as Report Target” in association with each of the plurality of pieces of representative waveform data output to the display apparatus D. In a case where operation information representing selection of any of a plurality of the checkboxes by the doctor is acquired by the first acquiring section 351, the output section 354 outputs, in the form of a report, representative waveform data corresponding to the selected checkbox as selected waveform data. For example, the report is electronic data obtained by putting together information that is required when the doctor gives explanation to a patient, when a request for consultation is made to a doctor of another medical institution, or when information is linked with an electronic medical record.

FIG. 10 depicts an example of output of a representative waveform data by the output section 354. When the first acquiring section 351 acquires, from the doctor's terminal 2, operation information representing selection, by the doctor, of a display area B1 of representative waveform data displayed in FIG. 9, the output section 354 outputs a display image in FIG. 10 to the display apparatus D. As represented by a rectangular broken line in FIG. 10, the output section 354 outputs the selected representative waveform data in a larger size on the left side.

In a case where the second acquiring section 352 acquires clear electrocardiogram data, each piece of which corresponds to a time interval corresponding to one beat, the output section 354 identifies one piece of or a predetermined number of pieces of fibrillation wave electrocardiogram data in descending order of proportions of clear electrocardiogram data included in original fibrillation wave electrocardiogram data before being divided into electrocardiograms each corresponding to a one-heartbeat unit. For example, each piece of the fibrillation wave electrocardiogram data corresponds to a 30-second time interval. For example, the predetermined number is designated in advance by the doctor, who is a user. The output section 354 outputs the identified fibrillation wave electrocardiogram data as representative waveform data.

For example, it is supposed that, in a case where the output section 354 determines whether or not fibrillation waves are clear every second about fibrillation wave electrocardiogram data corresponding to a time interval from 17:01:10.0 until 17:01:20.0 obtained by measurement performed for the patient U, the proportion of clear electrocardiogram data included in the fibrillation wave electrocardiogram data of the time interval is the second highest proportion of a plurality of pieces of fibrillation wave electrocardiogram data included in the same overall electrocardiogram data, and the predetermined number is three.

At this time, the output section 354 output three pieces of representative waveform data including the fibrillation wave electrocardiogram data corresponding to the time interval from 17:01:10.0 until 17:01:20.0 in descending order of the proportions of the clear electrocardiogram data included in the fibrillation wave electrocardiogram data. In this manner, since the output section 354 outputs one piece of or a predetermined number of pieces of fibrillation wave electrocardiogram data in descending order of the proportions at which clear electrocardiogram data is included, the doctor can diagnose a symptom of the patient while checking a plurality of heartbeats with clear fibrillation waves.

In a case where the second acquiring section 352 acquires, as clear electrocardiogram data, each piece of a plurality of pieces of fibrillation wave electrocardiogram data including heartbeats with clear fibrillation waves at a relatively high proportion, the output section 354 may output, as representative waveform data, one or more pieces of clear electrocardiogram data selected on the basis of the proportions of heartbeats with clear fibrillation waves included in the respective pieces of clear electrocardiogram data from the acquired plurality of pieces of clear electrocardiogram data. For example, the output section 354 identifies a plurality of pieces of clear electrocardiogram data acquired by the second acquiring section 352 as electrocardiogram data including heartbeats with clear fibrillation waves at a relatively high proportion, and further identifies, from the identified plurality of pieces of clear electrocardiogram data, clear electrocardiogram data in which heartbeats with clear fibrillation waves are included at a proportion which is equal to or higher than a threshold, the proportion being represented by a clear heartbeat index acquired along with the clear electrocardiogram data. For example, the threshold is a value set as a proportion by which even a person other than a cardiologist is expected to notice the presence of fibrillation waves. The output section 354 may output, as representative waveform data, a predetermined number of randomly selected pieces of clear electrocardiogram data from the identified clear electrocardiogram data. For example, the predetermined number is a value set by the doctor, who is a user of the doctor's terminal 2, via the accepting section 355.

The output section 354 may output, as representative waveform data, a predetermined number of pieces of clear electrocardiogram data selected in descending order of the proportions of heartbeats with clear fibrillation waves from a plurality of pieces of clear electrocardiogram data identified as electrocardiogram data in which heartbeats with clear fibrillation waves are included at proportions which are equal to or higher than a threshold. The output section 354 may output, as representative waveform data, a predetermined number of selected pieces of clear electrocardiogram data with the earliest measurement timings from clear electrocardiogram data in which heartbeats with clear fibrillation waves are included at proportions which are equal to or higher than a threshold.

### Process Performed at Time of Generation of Display Machine Learning Model

The explanation returns to FIG. 2. Hereinbelow, a process performed at the time of machine learning for generating the display machine learning model is explained as a process to be performed before the second acquiring section 352 acquires clear electrocardiogram data. The first acquiring section 351 acquires a plurality of pieces of electrocardiogram data for training which are a plurality of pieces of electrocardiogram data in which fibrillation waves are included. At this time, the first acquiring section 351 acquires a plurality of pieces of overall electrocardiogram data from the electrocardiograph 1 attached to each of a plurality of patients U. The first acquiring section 351 acquires a plurality of pieces of electrocardiogram data for training in which fibrillation waves are included, from the acquired plurality of pieces of overall electrocardiogram data by using the determination machine learning section 32 that classifies a plurality of pieces of electrocardiogram data into electrocardiogram data in which fibrillation waves are included, and electrocardiogram data in which fibrillation waves are not included.

Specifically, the first acquiring section 351 inputs overall electrocardiogram data to the determination machine learning model, and acquires a plurality of pieces of electrocardiogram data (hereinbelow, also referred to as electrocardiogram data for training) which is output by the determination machine learning model and in which fibrillation waves are included. The first acquiring section 351 generates sub-divided electrocardiogram data for training obtained by dividing the acquired electrocardiogram data for training into electrocardiograms each corresponding to a unit time. For example, the length of each unit time is time corresponding to one beat.

The accepting section 355 communicates with the doctor's terminal 2 via the communicating section 31. The accepting section 355 accepts an instruction for classification of a plurality of pieces of sub-divided electrocardiogram data for training into clear electrocardiogram data for training determined by the doctor as electrocardiogram data in which fibrillation waves are clear, and unclear electrocardiogram data for training determined by the doctor as electrocardiogram data in which fibrillation waves are included but are unclear.

More specifically, the accepting section 355 sequentially outputs the plurality of pieces of sub-divided electrocardiogram data for training acquired by the first acquiring section 351 to the display apparatus D of the doctor's terminal 2. From the doctor's terminal 2, the accepting section 355 accepts a result of decision by the doctor as to whether fibrillation waves in each of the sequentially-output plurality of pieces of sub-divided electrocardiogram data for training are clear or unclear. For example, the accepting section 355 accepts a decision result of determination by the doctor as to whether or not a fibrillation wave in each piece of the sub-divided electrocardiogram data for training obtained by dividing the electrocardiogram data for training into electrocardiograms each corresponding to time corresponding to one beat is clear. At this time, since it is sufficient if the doctor determines whether a fibrillation wave in each waveform corresponding to one beat is clear or unclear, the number of determination factors for determining whether or not a fibrillation wave is clear can be reduced as compared with a case where it is decided whether or not a fibrillation wave is clear in sub-divided electrocardiogram data for training obtained by dividing the electrocardiogram data into electrocardiograms each corresponding to time corresponding to two or more beats. Accordingly, since the accepting section 355 makes it easier for the doctor to determine whether or not a fibrillation wave is clear, determination errors can be reduced. Because of this, the accepting section 355 can enhance the precision of machine learning based on accepted results of determination by the doctor.

In examples in the present specification, the accepting section 355 receives results of decision by the doctor that electrocardiogram data in which the amplitude of fibrillation waves is within the predetermined range, a symptom of tachycardia is not observed, and noise with amplitude which is equal to or greater than the predetermined amplitude is not superimposed in frequencies higher than the frequency range of fibrillation waves is clear electrocardiogram data for training. On the other hand, the accepting section 355 receives results of decision by the doctor that electrocardiogram data in which the amplitude of fibrillation waves is smaller than the lower limit value of the predetermined range, electrocardiogram data in which the amplitude of fibrillation waves is greater than the upper limit value of the predetermined range, electrocardiogram data in which a symptom of tachycardia is observed, or electrocardiogram data in which noise with frequencies which are higher than the frequency range of fibrillation waves and with amplitude which is equal to or greater than the predetermined amplitude is superimposed is unclear electrocardiogram data for training.

The accepting section 355 stores, on the storage section 34, each of the plurality of pieces of sub-divided electrocardiogram data for training with a label representing a result of classification by the doctor as to whether the piece of the plurality of pieces of sub-divided electrocardiogram data for training is clear electrocardiogram data for training determined by the doctor as electrocardiogram data in which fibrillation waves are clear or unclear electrocardiogram data for training determined by the doctor as electrocardiogram data in which fibrillation waves are included but are unclear. Note that the present invention is not limited to the example in which the accepting section 355 accepts an instruction asking the doctor to perform classification as to whether or not electrocardiogram data is clear. For example, the accepting section 355 may accept an instruction asking a health care worker such as a clinical laboratory technician to perform classification as to whether or not electrocardiogram data is clear.

The determining section 353 may determine whether or not there is a signal in the frequency range corresponding to fibrillation waves by performing frequency analysis on the plurality of pieces of sub-divided electrocardiogram data for training generated by the first acquiring section 351. The determining section 353 may delete, from the storage section 34, electrocardiogram data for training determined as not including a signal in the frequency range corresponding to fibrillation waves. In this manner, the determining section 353 can prevent electrocardiogram data for training determined as not including a signal in the frequency range corresponding to fibrillation waves from being used for machine learning by the generating section 356.

The generating section 356 generates the display machine learning model that classifies a plurality of pieces of electrocardiogram data in which fibrillation waves are included into (i) electrocardiogram data in which fibrillation waves are clear, and (ii) electrocardiogram data in which fibrillation waves are included but are unclear. The generating section 356 generates the display machine learning model by performing machine learning using, as teaching data, a plurality of pieces of sub-divided electrocardiogram data for training labeled as clear electrocardiogram data for training and a plurality of pieces of sub-divided electrocardiogram data for training labeled as unclear electrocardiogram data for training.

In this manner, the generating section 356 can generate the display machine learning model mentioned above. Since, when the display machine learning model is generated, the electrocardiogram output system S transmits, to the doctor who checks whether fibrillation waves are clear, a plurality of pieces of electrocardiogram data in which fibrillation waves are included output by the determination machine learning model, the electrocardiogram output system S can reduce the number of pieces of electrocardiogram data to be checked by the doctor. Accordingly, the electrocardiogram output system S makes it possible to create the display machine learning model in a short time.

### Processing Procedure of Generation of Display Machine Learning Model

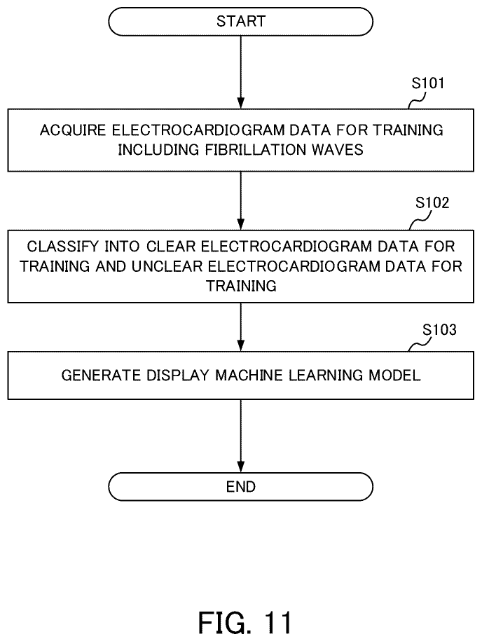

FIG. 11 is a flowchart depicting a processing procedure of generation of the display machine learning model by the output apparatus 3. For example, this processing procedure is started when the accepting section 355 accepts an instruction for generation of the display machine learning model from the doctor's terminal 2.

The first acquiring section 351 acquires a plurality of pieces of electrocardiogram data for training which are plurality of pieces of electrocardiogram data in which fibrillation waves are included (S101). The first acquiring section 351 generates sub-divided electrocardiogram data for training obtained by dividing the acquired electrocardiogram data for training into electrocardiograms each corresponding to a unit time. For example, the length of each unit time is time corresponding to one beat. The accepting section 355 accepts, from the doctor's terminal 2, an instruction for classification of a plurality of pieces of sub-divided electrocardiogram data for training into clear electrocardiogram data for training with clear fibrillation waves, and unclear electrocardiogram data for training with unclear fibrillation waves (S102).

In examples in the present specification, sub-divided electrocardiogram data for training in which the amplitude of fibrillation waves is within the predetermined range, a symptom of tachycardia is not observed, and predetermined noise is not superimposed is classified as clear electrocardiogram data for training. On the other hand, electrocardiogram data in which the amplitude of fibrillation waves is smaller than the lower limit value of the predetermined range, electrocardiogram data in which the amplitude of fibrillation waves is greater than the upper limit value of the predetermined range, electrocardiogram data in which a symptom of tachycardia is observed, or sub-divided electrocardiogram data for training in which predetermined noise is superimposed is classified as unclear electrocardiogram data for training.

The accepting section 355 stores, on the storage section 34, each of the plurality of pieces of sub-divided electrocardiogram data for training with a label representing a classification result of classification by the doctor as to whether the piece of the plurality of pieces of sub-divided electrocardiogram data for training is clear electrocardiogram data for training with clear fibrillation waves or unclear electrocardiogram data for training with unclear fibrillation waves. The generating section 356 generates the display machine learning model by performing machine learning using, as teaching data, a plurality of pieces of sub-divided electrocardiogram data for training labeled as clear electrocardiogram data for training and a plurality of pieces of sub-divided electrocardiogram data for training labeled as unclear electrocardiogram data for training (S103), and the process is ended.

### Processing Procedure of Output of Clear Electrocardiogram Data

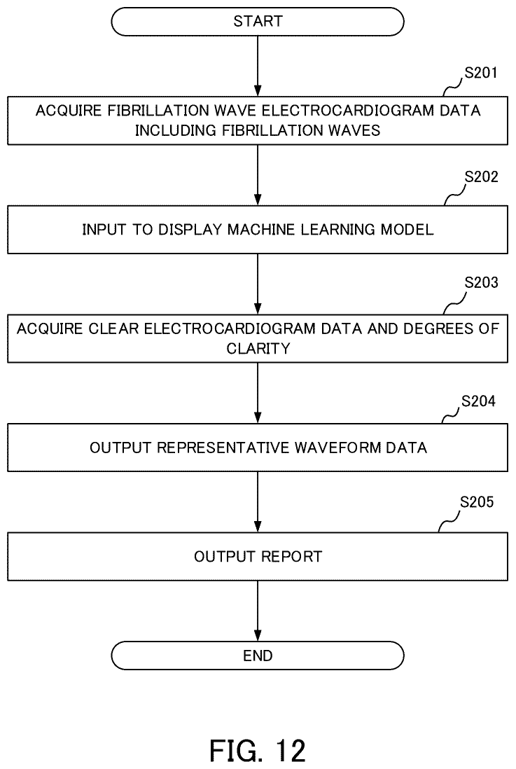

FIG. 12 is a flowchart depicting a processing procedure of output of clear electrocardiogram data by the output apparatus 3. This processing procedure is started when the first acquiring section 351 acquires, from the electrocardiograph 1, overall electrocardiogram data obtained by measuring temporal changes of an activity potential during an electrical activity of cardiac muscle.

The first acquiring section 351 acquires one or more pieces of fibrillation wave electrocardiogram data which is included in the acquired overall electrocardiogram data, and in which fibrillation waves are included (S201). The second acquiring section 352 generates sub-divided fibrillation wave electrocardiogram data obtained by dividing fibrillation wave electrocardiogram data into electrocardiograms each corresponding to a predetermined time unit. The second acquiring section 352 inputs one or more pieces of sub-divided fibrillation wave electrocardiogram data to the display machine learning section 33 that functions as the display machine learning model (S202), and acquires a plurality of pieces of clear electrocardiogram data output as electrocardiogram data in which fibrillation waves are clear from the display machine learning section 33, and the degree of clarity representing a degree of clarity of a fibrillation wave included in each of a plurality of pieces of clear electrocardiogram data (S203). The output section 354 outputs, as representative waveform data and to the doctor's terminal 2, a predetermined number of pieces of clear electrocardiogram data in descending order of the degrees of clarity from the plurality of pieces of clear electrocardiogram data acquired by the second acquiring section 352 (S204). The output section 354 outputs a report including representative waveform data selected by the doctor from the output plurality of pieces of representative waveform data (S205), and the process is ended.

### Advantages Achieved by Output Apparatus According to Present Embodiment

The second acquiring section 352 inputs the acquired plurality of pieces of sub-divided fibrillation wave electrocardiogram data to the trained display machine learning model, and acquires clear electrocardiogram data output by the display machine learning model as electrocardiogram data in which fibrillation waves are clear. At this time, the second acquiring section 352 outputs, as the clear electrocardiogram data, sub-divided fibrillation wave electrocardiogram data from the plurality of pieces of sub-divided fibrillation wave electrocardiogram data, the output sub-divided fibrillation wave electrocardiogram data being electrocardiogram data in which the amplitude of the fibrillation waves is within a predetermined range, a symptom of tachycardia is not observed, and predetermined noise is not superimposed. The output section 354 outputs the acquired clear electrocardiogram data to the doctor's terminal 2. In this manner, the output section 354 can make it possible for the doctor to grasp an electrocardiogram waveform representing grounds for the doctor to decide that the patient is having atrial fibrillation in her/his heart.

Whereas the present invention has been explained using embodiments thus far, the technical scope of the present invention is not limited by the scope of the description of the embodiments described above, but can be modified and changed variously within the scope of the gist. For example, all or some of apparatuses can be configured in a functionally or physically distributed/integrated manner in any units. In addition, embodiments of the present invention include also new embodiments that are generated by any combination of a plurality of embodiments. Advantages of the new embodiments generated by the combination combine advantages of the original embodiments.

What is claimed is:

1. An output method executed by a computer, the output method comprising:
a first acquisition step of acquiring a plurality of pieces of fibrillation wave electrocardiogram data including a fibrillation wave from overall electrocardiogram data obtained by measuring temporal changes of an activity potential during an electrical activity of cardiac muscle;
a second acquisition step of acquiring clear electrocardiogram data from the plurality of pieces of fibrillation wave electrocardiogram data acquired at the first acquisition step, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed; and
an output step of outputting the clear electrocardiogram data acquired at the second acquisition step.

2. The output method according to claim 1, wherein
the second acquisition step includes: inputting one or more pieces of the fibrillation wave electrocardiogram data acquired at the first acquisition step to a machine learning model trained such that, in a case where (i) electrocardiogram data in which a fibrillation wave is clear and (ii) electrocardiogram data in which a fibrillation wave is included but is unclear are input, the machine learning model outputs the electrocardiogram data in which a fibrillation wave is clear, and does not output the electrocardiogram data in which a fibrillation wave is included but is unclear; and acquiring, from the input one or more pieces of fibrillation wave electrocardiogram data, the clear electrocardiogram data output from the machine learning model.

3. The output method according to claim 2, wherein
the machine learning model is a machine learning model trained such that, in a case where the electrocardiogram data determined by a health care worker as electrocardiogram data in which a fibrillation wave is clear, and the electrocardiogram data determined by the health care worker as electrocardiogram data in which a fibrillation wave is included but is unclear are input, the machine learning model outputs the electrocardiogram data in which a fibrillation wave is clear, and does not output the electrocardiogram data in which a fibrillation wave is included but is unclear.

4. The output method according to claim 1, wherein the output step includes outputting one or more pieces of the clear electrocardiogram data selected from a plurality of pieces of the clear electrocardiogram data on a basis of a proportion of heartbeats with clear fibrillation waves in heartbeats included in each piece of the clear electrocardiogram data.

5. The output method according to claim 2, wherein
the output step includes: identifying one piece of or a predetermined number of pieces of fibrillation wave electrocardiogram data in descending order of proportions of heartbeats with clear fibrillation waves in heartbeats included in respective pieces of the clear electrocardiogram data; and outputting the identified piece or pieces of the identified fibrillation wave electrocardiogram data.

6. The output method according to claim 1, wherein
the first acquisition step includes acquiring, as the fibrillation wave electrocardiogram data and from a plurality of pieces of divided electrocardiogram data obtained by dividing the overall electrocardiogram data by date and time period of measurement of the overall electrocardiogram data, a plurality of pieces of the divided electrocardiogram data in which fibrillation waves are included, and
the output step includes outputting, on the display apparatus, a plurality of pieces of the clear electrocardiogram data whose dates or time periods of measurement are different from each other.

7. The output method according to claim 1, wherein
the output step includes: outputting, on a display apparatus, a plurality of pieces of representative waveform data selected on a basis of degrees of clarity of fibrillation waves from a plurality of pieces of the clear electrocardiogram data; and outputting, as selected waveform data, the representative waveform data selected by a user from the output plurality of pieces of representative waveform data.

8. The output method according to claim 7, wherein
the output step includes outputting the clear electrocardiogram data as the representative waveform data in descending order of the degrees of clarity of the fibrillation waves included in the acquired clear electrocardiogram data.

9. An output apparatus comprising:
a first acquiring section that acquires a plurality of pieces of fibrillation wave electrocardiogram data including a fibrillation wave from overall electrocardiogram data obtained by measuring temporal changes of an activity potential during an electrical activity of cardiac muscle;
a second acquiring section that acquires clear electrocardiogram data from the plurality of pieces of fibrillation wave electrocardiogram data acquired by the first acquiring section, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed; and
an output section that outputs the clear electrocardiogram data acquired by the second acquiring section.

10. A computer-readable storage medium that non-transitorily stores a program for causing a computer to execute:
a first acquisition step of acquiring a plurality of pieces of fibrillation wave electrocardiogram data including a fibrillation wave from overall electrocardiogram data obtained by measuring temporal changes of an activity potential during an electrical activity of cardiac muscle;
a second acquisition step of acquiring clear electrocardiogram data from the plurality of pieces of fibrillation wave electrocardiogram data acquired at the first acquisition step, the clear electrocardiogram data being electrocardiogram data in which amplitude of the fibrillation wave is within a predetermined range, a symptom of tachycardia is not observed, and noise in a frequency range which is higher than a frequency range of the fibrillation wave and with amplitude which is equal to or greater than predetermined amplitude is not superimposed; and
an output step of outputting the clear electrocardiogram data acquired at the second acquisition step.
